(19) United States
(12) Patent Application Publication
(10) Pub. No.: US 2017/0208486 A1
(43) Pub. Date: Jul. 20, 2017

(54) VOICE OPTIMIZATION ENABLEMENT APPARATUS

(71) Applicant: OPTULINK INC., NAPERVILLE, IL (US)

(72) Inventors: BRUCE M. PETERSON, SOMONAUK, IL (US); MICHELE D'AGOSTINO, SOUTHAMPTON (GB)

(21) Appl. No.: 15/416,540

(22) Filed: Jan. 26, 2017

Related U.S. Application Data
(63) Continuation of application No. PCT/US2015/042932, filed on Jul. 30, 2015.
(60) Provisional application No. 62/030,674, filed on Jul. 30, 2014.

(51) Int. Cl. H04W 24/02; H04L 12/823

(52) U.S. Cl. CPC H04W 24/02; H04L 47/32; H04W 24/08

(57) ABSTRACT

A control circuit operably couples to a plurality of network interfaces and receives, at least substantially in near real time, Internet Protocol (IP) flow content from a plurality of mobile communications (comprising at least voice communications). The control circuit analyzes that IP flow content to identify at least one mobile communications infrastructure cell that is underperforming with respect to voice quality and further identifies at least one cause for underperformance by that mobile communications infrastructure cell. The control circuit then enables voice optimization for that mobile communications infrastructure cell.

### CROSS REFERENCE TO RELATED APPLICATIONS

The present application is a U.S. Continuation Application which claims priority benefit under 35 U.S.C. §120 of co-pending International Patent Application No. PCT/US2015/042932, entitled “VOICE OPTIMIZATION ENABLEMENT APPARATUS,” filed Jul. 30, 2015; which application claims priority benefit from U.S. Provisional Patent Application No. 62/030,674, entitled “WCDMA VOICE OPTIMIZATION TRIGGER,” filed Jul. 30, 2014, co-pending at the date of filing; each of which, to the extent not inconsistent with the disclosure herein, is incorporated herein by reference.

### TECHNICAL FIELD

These teachings relate generally to the use of Internet Protocol flows to convey voice content.

### BACKGROUND

The use of Internet Protocol (IP)-based flows to convey voice information (for example, to support cellular telephony) comprises a known area of prior art endeavor. Because IP-based flows are packet-based, and further because of various dynamic vagaries in the signal chain, the quality of a voice message conveyed in this way can vary. The prior art has therefore also explored ways to attempt to respond to such circumstances. Many such approaches are generally viewed as pertaining to so-called mobile voice optimization.

In a typical prior art application setting, mobile voice optimization triggers are based upon Operation Support System (OSS)-generated cell key performance indicators (KPIs) and counters that are derived from mobile network elements (NE) or drive testing a given mobile network. Unfortunately, OSS cell performance KPIs and drive test-based approaches are not sufficiently granular, network-wide, real time or near real-time and inherently cannot provide information that is sufficiently timely to be genuinely successful at triggering voice optimization because voice quality in a mobile network tends to be highly temporally dynamic and often as a result of not-previously-detected cell congestion. Network probes can be more successful in these regards but tend to engender a very high corresponding cost and often require a very complicated and lengthy calibration and deployment effort and schedule.

Furthermore, prior art mobile voice optimization triggers typically cannot reliably properly trigger in the case of excessive packet error in the radio link caused by data overload bleeding over to voice channels in 3G voice applications and coverage and capacity events in 4G voice applications network wide in near real-time. In addition, prior art mobile voice optimization triggers cannot typically properly correlate in real-time between IP-based flows to convey voice information and OSS cell performance KPIs to trigger voice optimization.

### BRIEF DESCRIPTION OF THE DRAWINGS

The above needs are at least partially met through provision of the voice optimization enablement apparatus described in the following detailed description, particularly when studied in conjunction with the drawings, wherein:

FIG. 1 comprises a flow diagram as configured in accordance with various embodiments of these teachings.

FIG. 2 comprises a block diagram as configured in accordance with various embodiments of these teachings.

FIG. 3 comprises a process flow diagram as configured in accordance with various embodiments of these teachings.

FIG. 4 comprises a schematic block diagram as configured in accordance with various embodiments of these teachings.

FIG. 5 comprises a flow diagram as configured in accordance with various embodiments of these teachings.

FIG. 6 comprises a flow diagram as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, a control circuit operably couples to a plurality of network interfaces and receives, at least substantially in near real time, Internet Protocol (IP) flow content and/or OSS cell performance KPIs from a plurality of mobile communications (comprising at least voice communications). The control circuit analyzes that IP flow content and/or OSS cell performance KPIs to identify at least one mobile communications infrastructure cell that is underperforming with respect to voice quality and further identifies at least one cause for underperformance by that mobile communications infrastructure cell. The control circuit then enables voice optimization for that mobile communications infrastructure cell.

These teachings are highly flexible in practice and will serve compatibly with a variety of systems including 2G, 3G, 4G, GSM, UMTS, Wi-Fi and LTE systems. In all of these cases a single type of monitoring unit can reliably and effectively serve to detect voice optimization triggers.

By one approach the aforementioned IP flow content can comprise one or more of General Packet Radio Services (GPRS) Transport Protocol (GTP)v1 packets, GTPv2 packets, Wi-Fi packets, Real-time Transport Protocol packets, and/or Real-time Control Protocol packets. When the IP flow content includes GTPv1 packets, the aforementioned analysis can include analyzing the IP flow content to identify GTPv1 packets of mobile communications infrastructure cells with a top <X> traffic data volume per destination IP address and/or cell ID. As another illustrative example, when the IP flow content includes GTPv2 packets and/or Wi-Fi IP packets, the aforementioned analysis can include analyzing the IP flow content to identify mobile communications infrastructure cells with a worst <X> performance metric as regards at least one of Real-time Transport Protocol lapse and lossy packets.

By one approach, the control circuit analyzes the IP flow content to identify underperforming mobile communications infrastructure cells by forming corresponding metadata messages as a function of identifying mobile communications infrastructure cells exhibiting a worst performance metric. For example, when considering a mobile communications infrastructure cell that employs GTPv1 packets, the control circuit can form GTPv1-based metadata messages that are then transformed into at least one of volume, utilization rate, and throughput flat-line metrics. As another example, when considering a mobile communications infrastructure cell that employs GTPv2 and/or Wi-Fi IP packets, the control circuit can form GTPv2/Wi-Fi-based metadata messages that are then transformed into at least one of Real-time Transport Protocol lapse delay, jitter, latency, and Real-time Transport Protocol packet loss metrics.

By one approach, the control circuit identifies causes for underperformance by such a mobile communications infrastructure cell by, at least in part, using at least one OSS cell performance metric Key-Performance Indicator (KPI). Illustrative KPIs include but are not limited to bit error rate, interference, accessibility, and dropped call rate.

These teachings are highly flexible in practice and will accommodate a wide variety of application settings and parameters/indicators of interest. Such an approach can readily accommodate processing a large quantity of data and are highly suitable for use in triggering voice optimization in a mobile system in at least a near real-time manner. As a result voice communications can benefit from improved quality and system users can have a considerably improved user experience.

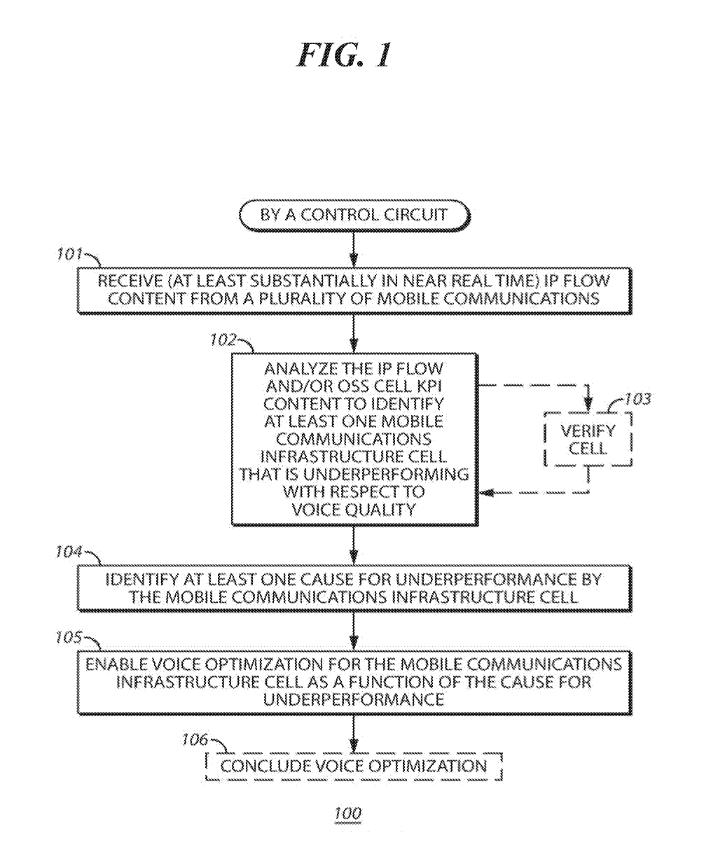

These and other benefits may become clearer upon making a thorough review and study of the following detailed description. Referring now to the drawings, and in particular to FIG. 1, an illustrative process 100 that is compatible with many of these teachings will now be presented.

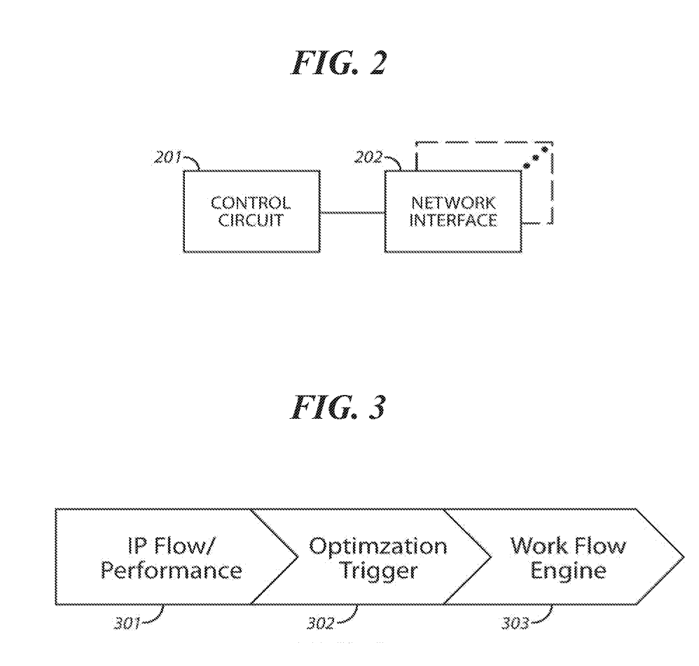

For the sake of an illustrative example it will be presumed here that a control circuit of choice carries out this process 100. Referring momentarily to FIG. 2, such a control circuit 201 can operably couple to a plurality of network interfaces 202. Being a “circuit,” the control circuit 201 therefore comprises structure that includes at least one (and typically many) electrically-conductive paths (such as paths comprised of a conductive metal such as copper or silver) that convey electricity in an ordered manner, which path(s) will also include corresponding electrical components (both passive (such as resistors and capacitors) and active (such as any of a variety of semiconductor-based devices) as appropriate) to permit the circuit to effect the control aspect of these teachings.

Such a control circuit 201 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 201 is configured (for example, by using corresponding programming as will be well understood by those skilled in the art) to carry out one or more of the steps, actions, and/or functions described herein.

By one approach this control circuit 201 includes integral digital memory. This memory can serve, for example, to non-transitorily store computer instructions that, when executed by the control circuit 201, cause the control circuit 201 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as an erasable programmable read-only memory (EPROM).)

The network interfaces 202 are operably coupled within a mobile communications network to receive IP flow and OSS cell performance KPI content as corresponds to a plurality of voice-conveying mobile communications.

These network interfaces 202 can include the Gn in a Universal Mobile Telecommunications Service (UMTS)/ High Speed Packet Access (HSPA) network, S1 and S11 in a Long Term Evolution (LTE) network, a Wi-Fi Gateway (GW) interface in the case of Wi-Fi, and an OSS interface for Network Element (NE)-generated KPIs. These voice-conveying mobile communications can be communications sourced by a mobile device (such as a cellular telephone or an automated voice system) and/or communications that are directed to a target mobile device. These teachings will accommodate a variety of voice-conveying packets including but not limited to General Packet Radio Services (GPRS) Transport Protocol (GTP)v1 packets, GTPv2 packets, Wi-Fi packets, Real-time Transport Protocol packets, and Real-time Control Protocol packets. (The conveyance of digitized voice content via a packet-based protocol comprises a well understood area of prior art endeavor. As the present teachings are not overly sensitive to the selection of any particular approach in these regards, further elaboration in these regards is not provided here for the sake of brevity.)

With continued reference to both FIGS. 1 and 2, at block 101 the control circuit 201 receives (at least substantially in near real-time) IP flow content and/or OSS cell performance KPIs from the aforementioned plurality of mobile communications via the plurality of network interfaces 202. (As used herein, the expression “near real-time” will be understood to refer to a time frame of less than 60 seconds for IP flow content and 15 minutes or some other duration less than an hour for OSS cell performance KPIs.

At block 102 the control circuit 201 analyzes the aforementioned IP flow content to identify at least one mobile communications infrastructure cell that is underperforming with respect to voice quality. To help ensure accurate results, by one approach this activity can include identifying and discarding duplicate IP flow content.

The specifics of this analysis can vary as desired to suit the specifics of a given application setting. When, for example, the IP flow content includes GTPv1 packets, this analysis can comprise, at least in part, identifying GTPv1 packets of mobile communications infrastructure cells with a top <X> traffic data volume per destination IP address and/or cell ID. Conversely, when the IP flow content includes GTPv2 and/or Wi-Fi IP packets, this can analysis can comprise, at least in part, identifying either of GTPv2 and/or Wi-Fi IP packets of mobile communications infrastructure cells with a worst <X> performance metric as regards at least one of Real-time Transport Protocol (RTP) lapses and RTP lossy packets.

In any event, the aforementioned analysis can include identifying mobile communications infrastructure cells with a worst <X> performance metric as regards at least one of delay, dropped calls, packet loss, latency, and jitter. These teachings can then also include forming metadata messages as a function of having so identified mobile communications infrastructure cells with a worst performance metric. For example, in the case of GTPv1 packets, the control circuit 201 can form GTPv1-based metadata messages as a function of identifying mobile communications infrastructure cells with a worst performance metric and then transform those GTPv1-based messages into at least one of a volume, utilization rate, or throughput flat-line metric. In the case of GTPv2 or Wi-Fi IP packets, the control circuit 201 can form GTPv2/Wi-Fi-based metadata messages as a function of identifying mobile communications infrastructure cells with a worst performance metric and then transforming those GTPv2/Wi-Fi-based messages into at least one of a Real-time Transport Protocol lapse delay, jitter, latency, or Real-time Transport Protocol packet loss metric.

By one optional approach, this process 100 will accommodate, at block 103, verifying the mobile communications infrastructure cell that is underperforming in these regards. This verification can include, for example, utilizing reference data to correlate cell identifiers to cell site identifiers and/or recording a last known cell identifier for a Wi-Fi location. Reference data may also include latitude and longitude information suitable to determine cell-to-cell distances. These teachings will accommodate other approaches in these regards as desired.

At block 104 the control circuit 201 identifies at least one cause for the detected underperformance by the identified mobile communications infrastructure cell. By one approach the control circuit 201 achieves this result, at least in part, by using at least one performance metric Key-Performance Indicator (KPI). Generally speaking, such a performance metric KPI can comprise one or more of bit error rate, interference, accessibility, and dropped call rate. More specifically, but without intending any specific limitations in these regards, the performance metric KPI can comprise one or more of VoLTE Call Drop Rate, HARQ DURATION, Hand Off (HO) Execution Failure Rate, Uplink received signal strength indicator (RSSI), and Hand Off Execution Failure Guard time.

These teachings are highly flexible in practice and will accommodate identifying any of a variety of underperformance causations. Examples in these regards include but are not limited to causes such as low accessibility, Uplink (UL) Received Signal Strength Indicator (RSSI) degradation, RRC connection failures due to timer expiry, physical layer failures, HO execution failures, high HO execution failures, HO execution guard failure, cell edge coverage, and VoLTE cell edge service degradation, to note but a few.

At block 105 the control circuit 201 then enables voice optimization for the mobile communications infrastructure cell as a function of the identified cause for underperformance. In particular, a particular kind of voice optimization suitable to address the cause of underperformance can be enabled. The voice optimization OSS parameters that correspond to underperformance causations in general are well understood in the art including but not limited to cell configuration parameters such as Reduce cellindividualoffsetEutran, Increase “b2Threshold1Utra”, Increase “PONomPusch” and “PONomPucch”, Increase “t304IntraLte”, to note a few. So configured, however, such a control circuit 201 permits a particular dynamic and likely momentary cause of underperformance as regards packet-based voice quality to be addressed at least in near real-time in a reliable and effective manner.

Since the cause of underperformance may in fact be only momentary, these teachings will optionally accommodate concluding such voice optimization as shown at block 106. This block 106 can include, for example, the control circuit 201 analyzing IP flow and KPIs subsequent to enabling voice optimization to determine when an event that impaired voice quality has abated. Upon detecting that abatement the control circuit 201 can conclude the previously-triggered voice optimization. By one approach, for example, the control circuit 201 stores pre-optimization cell configuration parameters before optimization and reverts to those stored pre-optimization cell configuration parameters upon detecting the aforementioned abatement.

There is also the possibility that attempts at voice optimization might worsen an already impaired voice quality. In that case, this activity can also include detecting when relevant voice quality IP flow and/or OSS KPIs degrade following optimization in which case the control circuit 201 can revert to the stored pre-optimization cell configuration parameters.

To further illustrate the foregoing teachings a more specific implementation example will now be provided. It shall be understood that no particular limitations are intended by way of the specifics of this example.

FIG. 3 generally illustrates activity flow as per this illustrative example. An IP flow record analyzer component 301 assesses IP flow and performance and sends corresponding metadata representing the top traffic in the case of GTPv1 packets and/or RTP lapse or lossy packet flows in the case of GTPv2 and/or Wi-Fi packets to an optimization triggering component 302. The optimization triggering component 302 receives those metadata messages and transforms the messages into poor-voice-quality cell indicators, determines the corresponding metrics, compares those metrics to a set of preset optimization targets, and triggers corresponding cell sites for optimizations when the target threshold is exceeded. In this example the work flow engine 303 identifies/verifies the root cause for the poor voice quality using radio access network (RAN) KPIs and makes a corresponding decision regarding how to optimize the target cell(s).

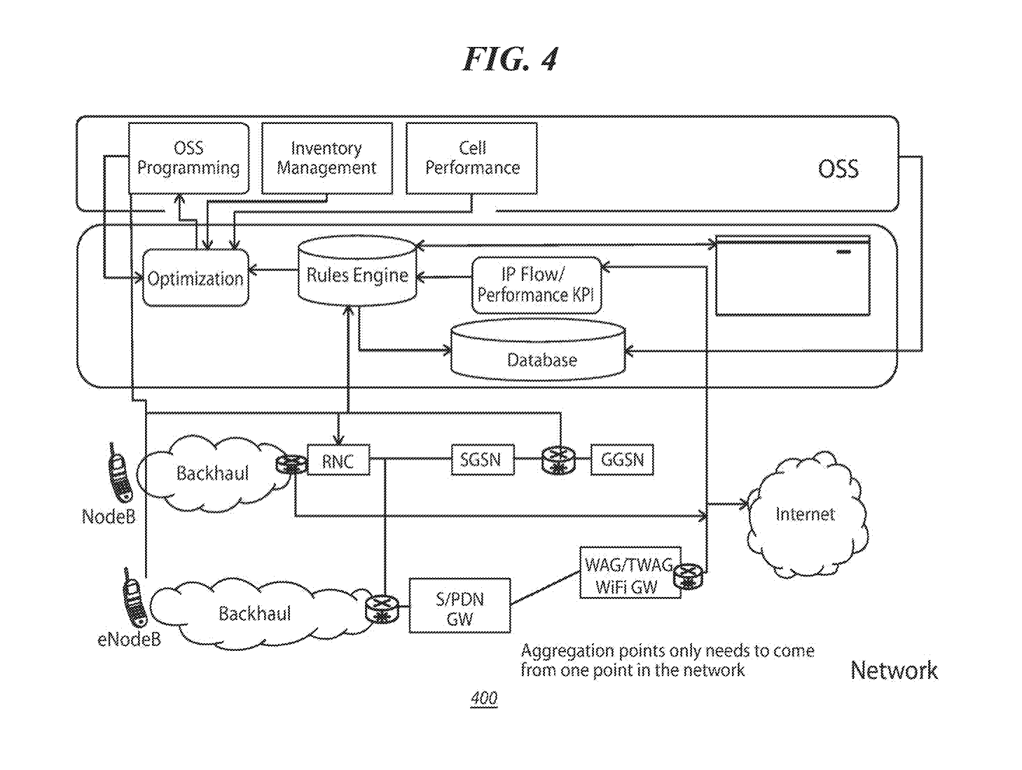

FIG. 4, in turn, illustrates an example of a system 400 having a network architecture configured to serve in the foregoing regards. Components illustrated in FIG.4 include tapping or mirroring IP flow interfaces at an aggregation point in front of the Radio Network Controller (RNC) facing the NodeBs in the case of a UMTS/HSPA network, in front of the Serving Gateway (S/PDN) facing the eNodeBs in the case of an LTE network, and/or tapping the interface between a Wi-Fi Gateway and the Internet in the case of Wi-Fi. The IP flow interfaces connect to an IP Flow/ Performance KPI Collector that together with a Rules Engine determines Worst <X> cells to trigger optimizations. An Optimization engine utilizes OSS interfaces to retrieve OSS cell performance KPIs from a cell performance component, reference data from an Inventory Management component which provide, for example, cell ID information, NodeB ID information, eNodeB ID information, and corresponding latitude/longitude information), and utilizes an OSS provisioning component (which can include, for example, a configuration management interface) that allows retrieval of parameter information and the ability to change or set parameters on a cell-level basis.

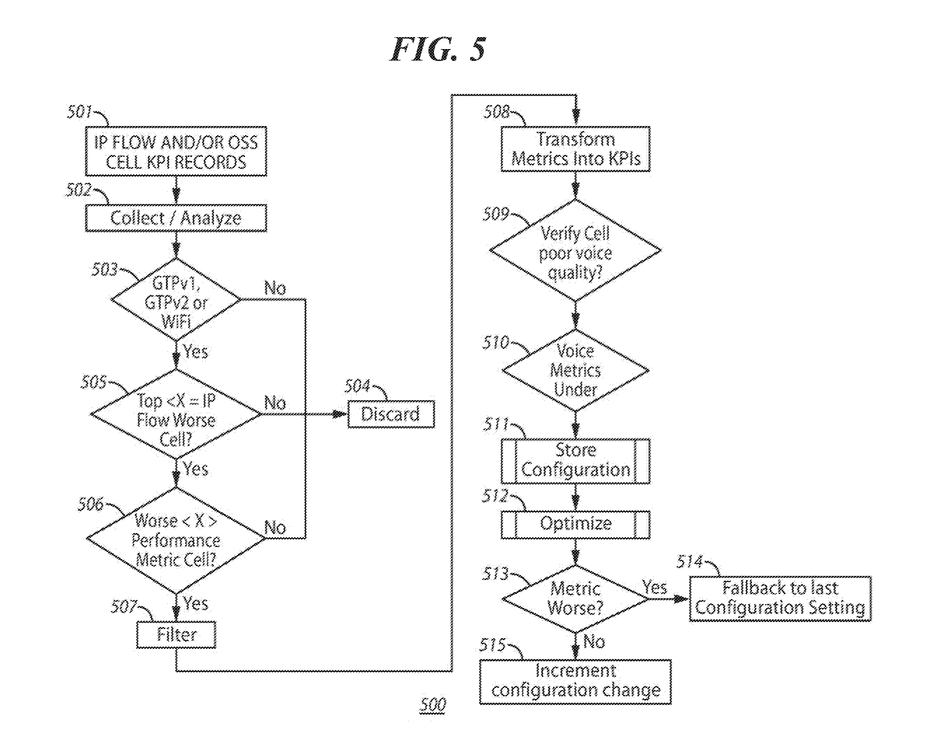

FIG. 5 presents a process 500 to be carried out by the above-described system 400.

At blocks 501 and 502 IP flow and/or OSS cell performance KPI records of interest are collected and analyzed. The IP flow records are provided from edge and/or core routers in the mobile backhaul and/or in the backbone network shown in FIG. 4. The OSS cell performance KPI records are retrieved from the OSS component shown in FIG. 4. By one approach a preliminary analysis determines whether these collected materials include any duplicate IP flow or OSS cell performance KPI records. Duplicate records are discarded in this example.

At decision block 503, records that do not pertain to GTPv1, GPTv2, or Wi-Fi are discarded 504. At decision block 505, GTPv1 IP flow records of cells with the top <X> traffic data volume per destination IP address (host) and/or cell ID for GTPv2 and/or Wi-Fi IP flow records of cells with the top <X> RTP lapses and/or lossy packets are identified with any remaining content being discarded at block 504.

At decision block 506 this process 500 provides for identifying cells using IP flow and/or OSS cell performance KPI records with the worst <X> performance metrics as regards highest delay, dropped calls, packet loss, and/or jitter. Remaining content is discarded at block 504.

At block 507 the IP flow and/or OSS cell performance KPI content meeting the aforementioned analysis is filtered based on cell and transform metrics. Those metrics are then conveyed as metadata in messages that are sent (using, for example, Syslog) to the aforementioned optimization trigger 302.

The optimization trigger 302 receives the foregoing messages and at block 508 transforms those metrics into key-performance indicators. For example, GTPv1-based messages are transformed into volume, utilization rate, and/or throughput flat-line KPIs and GTPv2 and Wi-Fi-based messages are transformed into RTP lapse delay, jitter, and RTP packet loss KPIs. The aforementioned utilization rate can be determined, for example, by dividing throughput by cell capacity. The aforementioned RTP lapse delay can be determined, for example, by counting the number of seconds RTP packets are missing as a reliability KPI. An example of the formula for RTP packet lapse is:

$$Audio Gaps ( \% ) = \frac{\# calls with audio gap instances}{Total \# two minutes calls} .$$

And the aforementioned RTP packet loss can be determined, for example, by counting the number of consecutive missing RTP packets and using that number as a voice quality KPI. For example, three or four consecutive missing RTP packets may be treated as a negative voice quality KPI. An example of the formula for RTP packet lapse is:

$$Acceptable Voice quality ( \% ) = 1 - \frac{\# seconds of speech affected by packet burst}{( Total \# seconds of voice call )} .$$

At decision block 509 poor voice quality at a particular cell is verified. This verification can include utilizing reference data to correlate the cell ID to a cell site ID or utilizing a last known recorded cell ID for Wi-Fi location. By one approach verifying the cell location of IP flow data such as RTP packets includes analyzing both the RTP stream packets and GTPv1 messages to capture the cell ID used to determine location. Generally speaking, this comprises correlating IP flow (such as an RTP stream)-based metrics to OSS cell performance KPIs in near real time to determine poor voice reliability and quality to thereby qualify a cell for voice optimization.

At block 510 this process 500 provides for determining if the cell voice quality is under an acceptable quality threshold. This activity can also include identifying a root cause that underlies the detected poor performance. That root cause can be identified, at least in part, by referring to OSS cell performance metric KPIs described in detail above. Put another way, this step provides for using OSS cell performance metric KPIs to determine both whether cell voice quality is under a predetermined threshold and to further identify one or more causative factors that are instigating that poor voice quality.

At block 511 this process provides for storing existing NodeB and/or eNodeB cell configuration parameters before conducting voice optimization. Then, at block 512, voice optimization is enabled by changing NodeB and/or eNodeB cell Radio Resource Management (RRM) configuration parameters based on the previously determined root cause(s).

At decision block 513 the process provides for determining whether the voice quality IP flow or corresponding OSS cell performance metric KPIs degraded. In other words, whether the effort to optimize voice led to an opposite result. When true, at block 514 this process 500 provides for falling back to the last configuration setting for the cell.

Otherwise, at block 515 this process will accommodate incrementing further changes to the configuration to attempt to further improve voice quality.

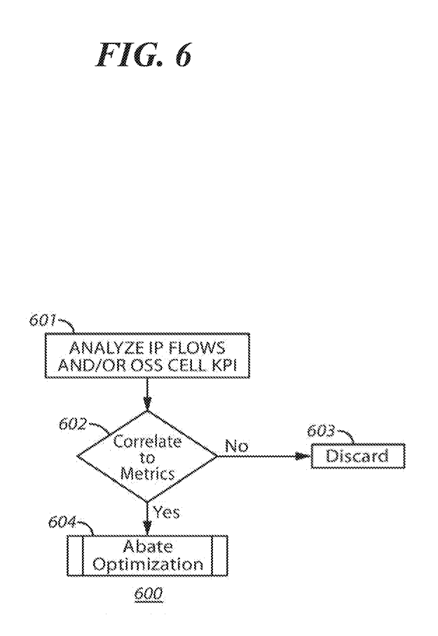

With reference to FIG. 6, a corollary process 600 provides at block 601 for analyzing the IP flows (and OSS cell performance KPIs if desired) to determine if the causation event that impaired voice quality has abated. This analysis can include a determination at block 602 regarding a correlation of that incoming data to one or more metrics of choice. Uncorrelated content can be discarded 603. Correlations, however can trigger abatement of voice optimization at block 604.

So configured, these teachings provide an optimization trigger that can leverage a single type of monitoring system to detect poor voice quality cell sites for a variety of telephony technologies including 3G, Wi-Fi, 4G, and even 5G. The disclosed teachings provide a voice optimization trigger that properly triggers in the case of excessive packet error in the radio link caused by data overload bleeding over to voice channels in 3G voice applications and coverage and capacity events in 4G voice applications network wide in near real time.

Furthermore, the disclosed teachings provide mobile voice optimization triggers that are properly correlated in real-time between IP-based flows to convey voice information and OSS cell performance KPIs that properly trigger mobile voice optimization.

What is more, the disclosed optimization trigger can reliably detect poor voice quality cell sites leveraging only limited interfacing with operations, administration, and maintenance systems as well as operational support systems, thereby avoiding considerable complexity and corresponding expense. Those skilled in the art will appreciate that these benefits are realized without requiring network interface probes and their corresponding analyzers, relying instead on a relatively simple IP flow record analyzer. It will also be appreciated that, although these teachings leverage some basic OSS cell performance KPI information, the disclosed approaches reduce any need to collect detailed OSS cell performance KPI information from different kinds of interfaces, hence avoiding the complexity and cost of supporting those interfaces and avoiding any need to adopt KPI interface changes when the corresponding vendor makes changes to the interface and/or any corresponding file formats.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

What is claimed is:

1. An apparatus comprising:
a plurality of network interfaces;
a control circuit operably coupled to the plurality of network interfaces and configured to:
receive, via the plurality of network interfaces and at least substantially in near real time, Internet Protocol (IP) flow content from a plurality of mobile communications;
analyze the IP flow content to identify at least one mobile communications infrastructure cell that is underperforming with respect to voice quality;
identify at least one cause for underperformance by the mobile communications infrastructure cell; and
enable voice optimization for the mobile communications infrastructure cell as a function, at least in part, of the cause for underperformance by the mobile communications infrastructure cell.

2. The apparatus of claim 1 wherein the IP flow and/or OSS cell performance metric KPI content comprises at least one of:
General Packet Radio Services (GPRS) Transport Protocol (GTP)v1 packets;
GTPv2 packets;
Wi-Fi packets;
Real-time Transport Protocol packets;
Real-time Control Protocol packets;
OSS cell performance metric KPIs.

3. The apparatus of claim 1 wherein the control circuit is configured to analyze the IP flow content, at least in part, by identifying and discarding duplicate IP flow content.

4. The apparatus of claim 1 wherein the control circuit is configured to analyze the IP flow content, at least in part, by identifying Group Transport Protocol (GTP)v1 packets of mobile communications infrastructure cells with a top<X> traffic data volume per destination IP address and/or cell ID.

5. The apparatus of claim 1 wherein the control circuit is configured to analyze the IP flow content, at least in part, by identifying either of General Packet Radio Services (GPRS) Transport Protocol (GTP)v2 and/or Wi-Fi IP packets of mobile communications infrastructure cells with a worst <X> performance metric as regards at least one of Real-time Transport Protocol (RTP) packet lapses and RTP lossy packets.

6. The apparatus of claim 1 wherein the control circuit is configured to analyze the IP flow and/or OSS cell performance metric KPI content, at least in part, by identifying mobile communications infrastructure cells with a worst <X> performance metric as regards at least one of delay, dropped calls, packet loss, latency, and jitter.

7. The apparatus of claim 6 wherein the control circuit is configured to analyze the IP flow content to identify at least one mobile communications infrastructure cell that employs GTPv1 packets and that is underperforming with respect to voice quality, at least in part, by:
forming GTPv1-based metadata messages as a function of identifying mobile communications infrastructure cells with a worst performance metric;
transforming the GTPv1-based messages into at least one of volume, utilization rate, and throughput flat-line metrics.

8. The apparatus of claim 6 wherein the control circuit is configured to analyze the IP flow content to identify at least one mobile communications infrastructure cell that employs at least one of GTPv2 and Wi-Fi IP packets and that is underperforming with respect to voice quality, at least in part, by:
forming GTPv2/Wi-Fi-based metadata messages as a function of identifying mobile communications infrastructure cells with a worst performance metric;
transforming the GTPv2/Wi-Fi-based messages into at least one of Real-time Transport Protocol packet lapse, delay, jitter, latency, and Real-time Transport Protocol packet loss metrics.

9. The apparatus of claim 6 wherein the control circuit is further configured to verify cells, at least in part, by at least one of:
utilizing reference data to correlate cell identifiers to cell site identifiers; and
recording a last known cell identifier for a Wi-Fi location.

10. The apparatus of claim 1 wherein the control circuit is configured to identify at least one cause for underperformance by the mobile communications infrastructure cell by, at least in part, using at least one OSS cell performance metric Key-Performance Indicator (KPI).

11. The apparatus of claim 10 wherein the OSS cell performance metric KPI comprises at least one of:
bit error rate;
interference;
accessibility;
dropped call rate.

12. The apparatus of claim 10 wherein the OSS cell performance metric KPI comprises at least one of Hybrid Automatic Repeat Request (HARQ) Duration, Hand Off (HO) Execution Failure Rate, UpLink (UL) Received Signal Strength Indicator (RSSI), HO Exec Fail Guard timer, and Radio Bearer Releases.

13. The apparatus of claim 1 wherein the control circuit is configured to identify at least one cause for underperformance by the mobile communications infrastructure cell by, at least in part, identifying the at least one cause as being at least one of:
Low Accessibility;
UL RSSI Degradation;
RRC Connection Failures due to timer expiry;
Physical Layer Failures;
HO Execution Failure;
High HO Execution Failure;
HO Execution Guard Failure;
Cell edge coverage;
VoLTE Cell Edge Service Degradation.

14. The apparatus of claim 1 wherein the control circuit is configured to enable voice optimization for the mobile communications infrastructure cell by first storing pre-optimization cell configuration parameters before optimization.

15. The apparatus of claim 14 wherein the control circuit is configured to enable voice optimization for the mobile communications infrastructure cell by changing or recommending the change of at least one OSS cell configuration parameter comprised of but not limited to:
reduce cellindividualoffsetEutran;
increase “b2Threshold1Utra”;
increase “PONomPusch” and “PONomPucch”;
increase “t304IntraLte”.

16. The apparatus of claim 14 wherein the control circuit is further configured to:
analyze IP flow and OSS cell performance metric key-performance indicators subsequent to voice optimization to determine when an event that impaired voice quality has abated;
upon determining that the event has abated, halting voice optimization and reverting to the stored pre-optimization cell configuration parameters by reverting to the stored pre-optimization cell configuration parameters when at least one of voice quality IP flow and/or OSS cell performance metric key-performance indicators degrade following optimization.
